(19) United States
(12) Patent Application Publication
(10) Pub. No.: US 2007/0249549 A1
(43) Pub. Date: Oct. 25, 2007

(54) Compounds and Methods for Rna Interference of the P65 Subunit of Nf-Kappa-B

(75) Inventors: Lars-Goran Axelsson, Oriongatan (SE); Liam Good, Sveavagen (SE)

Correspondence Address: MORRISON & FOERSTER LLP, 425 MARKET STREET, SAN FRANCISCO, CA, 94105-2482, US

(73) Assignee: Index Pharmaceuticals AB, Stockholm (SE)

(21) Appl. No.: 10/582,946

(22) PCT Filed: Dec. 17, 2004
(86) PCT No.: PCT/SE04/01911

§ 371 Date: Mar. 2, 2007

Related U.S. Application Data
(60) Provisional application No. 60/529,765, filed on Dec. 17, 2003.

(30) Foreign Application Priority Data
Dec. 17, 2003 (SE) 0303397-4

(51) Int. Cl. A61K 31/7125; C07H 21/02; C12N 15/63; C12N 5/06; C12N 5/10

(52) U.S. Cl. 514/44; 435/320.1; 435/325; 435/366; 536/24.1

(57) ABSTRACT

This invention relates to compounds, compositions, and methods useful for modulating the expression and activity of NF-kappa-B by RNA interference (RNAi) using small nucleic acid molecules, such as short interfering nucleic acid (siNA), short interfering RNA (siRNA) and double-stranded RNA (dsRNA). Furthermore the invention provides methods for preventing, treating or alleviating NF-kappa-B dependent diseases whereby NF-kappa-B is believed to play a role in the pathogenesis of a disease in a subject, preferably a human, by administration of a therapeutic effective and in a pharmacologically accepted form, the siRNA compounds of the invention.

### FIELD OF THE INVENTION

The present invention concerns compositions and methods useful in modulating gene expression associated with inflammation and allergic responses. More specifically, the invention relates to short interfering nucleic acid molecules (siRNA) capable of mediating RNA interference (RNAi) against the p65 subunit of the transcription factor NF-kappa-B, as well as pharmaceutical compositions thereof and methods for their use as a therapeutic for the treatment of inflammatory and allergic type diseases.

### BACKGROUND OF THE INVENTION

Nuclear factor kappa B (NF-kappa-B) is a member of the Rel/NF-kappa-B family of inducible pleiotropic transcription factors that play a pivotal role in a wide array of physiological and pathological responses including immune modulation, inflammatory responses, cancer and apoptosis. This extraordinary degree of involvement results from the ability of such inducible transcription factors to control the expression of a large multitude of various key genes involved in cellular processes. Consequently, there has been intense scientific activity in the NF-kappa-B field that has provided increasing evidence that NF-kappa-B is a major, if not the major transcription factor regulating inflammation and immunity.

In mammals the Rel/NF-kappa-B family consists of some 5 identified proteins, NF-kappa-B1 (p50 & precursor protein p105), NF-kappa-B2 (p52 & precursor protein p100), p65 (RelA), c-Rel, and RelB. The most prevalent form of NF-kappa-B heterodimer consists of a p50 subunit and a p65 subunit and is found in the cytoplasm of most cell types. By convention, any homo- or heterodimer is termed NF-kappa-B. The inactive NF-kappa-B dimmer is present in the cytosol bound to inhibitory proteins termed inhibitor protein I-kappa B (IκB), to which there are seven known types, the most important being I-kappa-B-alpha and I-kappa-B-beta.

Activators of NF-kappa B, such as lipopolysaccharide (LPS) and TNF-alpha, induce site-specific phosphorylation of I-kappa B and consecutive rapid dissociation of the complex accompanied by proteolytic degradation of I-kappa B. The released NF-kappa-B subsequently transmigrates from the cytosol into the nucleus where it binds to specific sequence elements and activates the transcription of a whole multitude of diverse genes with diverse functions.

Many pro-inflammatory cytokine genes have NF-kappa-B binding sites, and as a result inhibition of NF-kappa-B driven transcription is likely to be a pivotal element in the pathogenesis of various inflammatory type diseases, cancer, immune modulation and apoptosis. Some of these induced proteins can in turn activate NF-kappa-B leading to the further amplification and perpetuation of the inflammatory response. Recently, NF-kappa-B has been shown to have an anti-apoptotic role in certain cell types, most likely by inducing the expression of anti-apoptotic genes. This function may protect tumor cells against anti-cancer treatments and opens the possibility to use NF-kappa-B inhibiting compounds to sensitize the tumor cells and to improve the efficiency of the anti-cancer treatment.

Due to NF-kappa-B's direct role in regulating responses to inflammatory cytokines, it is perhaps not surprising that it plays an important role in the development of various diseases such as chronic inflammatory diseases such as rheumatoid arthritis, asthma and inflammatory bowel disease; acute diseases such as septic shock; Alzheimer's disease where the ss-amyloid protein activates NF-KB; atherosclerosis, where NF-kappa-B may be activated by oxidized lipids; autoimmune disease such as systemic lupus erythematosus; cancer by up-regulating certain oncogenes or by preventing apoptosis. In addition, NF-kappa-B is also involved in viral infection since NF-kappa-B is activated by different viral proteins, such as occurs upon infection with rhinovirus, influenza virus, Epstein-Barr virus, HTLV, cytomegalovirus or adenovirus. Furthermore, several viruses such as HIV have NF-kappa-B binding sites in their promoter/enhancer regions. Because of the potential role of NF-kappa-B in many of the above-mentioned diseases, NF-kappa-B and its regulators have drawn much interest as targets for the treatment of NF-KB related diseases, Glucocorticoids are effective inhibitors of NF-kappa-B, but they have endocrine and metabolic side effects when given systematically. Antioxidants may represent another class of NF-kappa-B inhibitors, but currently available antioxidants such as acetylcysteine are relatively weak and unspecific. However many compounds that have demonstrated inhibitory properties against NF-kappa-B have not been successfully developed as potential drugs due to the often serious nature of unwanted effects.

Consequently there is an obvious need for new compounds that are able to achieve effective and specific inhibition of NF-kappa-B without being compromised by serious unwanted side effects. Such compounds are the subject of this application.

RNA Interference

The following is a discussion of relevant art pertaining to RNA interference (RNAi). The discussion is provided only for understanding of the invention that follows. The summary is not an admission that any of the work described below is prior art to the claimed invention.

RNAi is a sequence-specific gene silencing process induced by double-stranded RNA (dsRNA). RNAi is a natural mechanism, which can also be used to provide information about gene function quickly, easily, and inexpensively. The use of RNAi for genetic-based therapies is widely studied, especially in viral infections, cancers, and inherited genetic disorders. RNAi has been used to make tissue-specific knockdown mice for studying gene function in a whole animal. Combined with genomics data, RNAi-directed gene silencing could allow functional determination of any gene expressed in a cell or pathway.

The term “RNA interference” (RNAi) was first coined after the discovery that injection of dsRNA into the nematode C. elegans leads to specific silencing of genes highly homologous in sequence to the delivered dsRNA (Fire et al., 1998, Nature, 391,806). RNAi was subsequently later observed to function in insects, frogs, and other animals including mice.

RNA interference (RNAi) describes a phenomenon wherein double-stranded RNA (dsRNA), when present inside a cell, inhibits expression of an edogenous gene that has an identical or nearly identical sequence to that of the dsRNA. Inhibition is caused by the specific degradation of the messenger RNA (mRNA) transcribed from the target gene. In greater detail, RNA interference describes a process of sequence-specific post-transcriptional gene silencing in animals mediated by so called “short interfering RNAs” (siRNAs) (Fire et al., 1998).

The natural function of RNAi appears to be protection of the genome against invasion by mobile genetic elements such as retrotransposons and viruses which produce aberrant RNA or dsRNA in the host cell when they become active (Jensen et al., 1999; Ketting et al., 1999; Ratcliff et al., 1999; Tabara et al., 1999). The process of post-transcriptional gene silencing is therefore believed to be an evolutionarily-conserved cellular defense mechanism present in the majority of mammalian cell types and is used to prevent the expression of foreign genes such as those derived from infection of viruses. This assumption is further strengthened by the observation that RNAi in animals, and the related phenomena of Post-transcriptional gene silencing (PTGS) in plants, result from the same highly conserved mechanism, indicating an ancient origin.

The basic process involves a dsRNA that is processed by cleavage into shorter units (called short interfering RNA; siRNA) that guide recognition and targeted cleavage of homologous target messenger RNA (mRNA).

The currently known mechanism of RNAi can be described as follows:

The processing of dsRNA into siRNAs, which in turn induces degradation of the intended target mRNA, is a two-step RNA degradation process. The first step involves a dsRNA endonuclease (ribonuclease III-like; RNase III-like) activity that processes dsRNA into smaller sense and antisense RNAs which are most often in the range of 21 to 25 nucleotides (nt) long, giving rise to the so called short interfering RNAs (siRNAs). This RNase III-type protein is termed “Dicer”. In a second step, the antisense siRNAs produced combine with, and serve as guides for, a different ribonuclease complex called RNA-induced silencing complex (RISC), which cleaves the target homologous single-stranded mRNAs. Cleavage of the target mRNA has been observed to place in the middle of the duplex region complementary to the antisense strand of the siRNA duplex and the intended target mRNA.

Inhibition of gene expression using siNA or siRNA has also been described in recently published patent applications. WO 03/070970 relates to RNAi mediated inhibition of NF-Kappa-B gene expression using siNA or siRNA. WO 03/070918 relates general to inhibition of gene expression using chemically modified siNA. Both WO 03/070970 and WO 03/070918 describe a large amount of theoretically possible target, sense and antisense sequences. However, in WO 03/070970 no evidence that demonstrates that the systems described in the applications actually works is presented. For example, in vitro experiments are carried out in cell free systems to measure siRNA activity in a luciferase assay. There are no evidences that the selected siNA or siRNA molecules inhibit the expression of the NF-kappa-B in vivo in an animal or a human. Although there are methods for computational prediction of potential target sites it is important to evaluate the sense and antisense sequences both in vitro and in vivo to acquire credibility of the suggested system.

Other reports showing the efficacy of RNAi in inhibiting the expression of the NF-Kappa-gene, in particular the p65 subunit of said gene have been published, such as Surabhi R. M. and Gaynor R. B., 2002; Zhou A. et al., 2003; Savage J. et al., 2003; and WO 03/020754.

While these studies and others indicate that there are certain requirements that need to be fulfilled in order to mediate efficient RNAi activity, such as length of the RNAi as measured in nucleotide bases, structure, chemical composition, and indeed even the sequence, there is no general agreement as to the characteristics of an effective RNAi construct. The issues of specificity, efficacy, and side effects need to be handled on a case-by-case basis.

Chemical modifications have been addressed through the work of Kreutzer et al., (see the published international patent application WO 00/44895) describing certain chemical modifications for use in dsRNA constructs in order to prevent activation of double-stranded RNA-dependent protein kinase PKR, specifically 2′-amino or 2′-O-methyl nucleotides, and nucleotides containing a 2′-O or 4′-C methylene bridge.

Longer dsRNA have also been the subject of investigations, as for example in the work of Beach et al., see the published international patent application WO 01/68836. The authors describe specific methods for blocking gene expression using endogenously-derived dsRNA.

### SUMMARY OF THE INVENTION

This invention relates to compounds, compositions, and methods useful for modulating the expression and activity of NF-kappa-B by RNA interference (RNAi) using small nucleic acid molecules, such as short interfering nucleic acid (siNA), short interfering RNA (siRNA) and double-stranded RNA (dsRNA) as specified in the attached claims, incorporated herein by reference.

Furthermore the invention provides methods for preventing, treating or alleviating NF-kappa-B dependent diseases whereby NF-kappa-B is believed to play a role in the pathogenesis of a disease in a subject, preferably a human, by administration of a therapeutic effective and in a pharmacologically accepted form, the siRNA compounds of the invention.

### BRIEF DESCRIPTION OF FIGURES

The invention will be described in closer detail in the following description, examples, and attached drawings, in which

FIG. 1 shows the activity of endogenous NF-kappa-B in cells transfected with 4 siRNA compounds according to the invention;

FIG. 2 shows the NF-kappa-B p65 subunit protein levels in 293 cells transfected with siRNA compounds; and

FIG. 3 shows the results of Example 4 in the form of a histogram with different physiological criteria used to assess improvement in the degree of inflammation of the gastrointestinal tract after administration of a siRNA compound.

### DESCRIPTION

Before the present method is disclosed and described, it is to be understood that this invention is not limited to the particular configurations, process steps, and materials disclosed herein, as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

Definitions

In describing, exemplifying and claiming the present invention, the following terminology will be used in accordance with the definitions set out below. Where not otherwise indicated, the terms are intended to have the meaning generally recognized in the art.

By “p65 subunit of NF-kappa-B” is meant any polynucleotide encoding the p65 subunit of NF-kappa-B.

By “p65 subunit of NF-kappa-B protein” is meant any p65 subunit of NF-kappa-B peptide or protein or a component thereof, wherein the peptide or protein is encoded by the p65 subunit of NF-kappa-B gene.

By “highly conserved sequence region” is meant a nucleotide sequence of one or more regions in a target gene, which does not vary significantly from one generation to the other or from one biological system to the other.

By “sense region” is meant a nucleotide sequence of a siNA molecule having complementarity to an antisense region of the siNA molecule. In addition, the sense region of a siNA molecule can comprise a nucleic acid sequence having homology with a target nucleic acid sequence.

By “antisense region” is meant a nucleotide sequence of a siNA molecule having complementarity to a target nucleic acid sequence. In addition, the antisense region of a siNA molecule can optionally comprise a nucleic acid sequence having complementarity to a sense region of the siNA molecule.

By “target nucleic acid” is meant any nucleic acid sequence whose expression or activity is to be modulated. The target nucleic acid can be DNA or RNA.

By “complementarity” is meant that a nucleic acid can form hydrogen bond(-s) with another nucleic acid sequence by either traditional Watson-Crick or other non-traditional types or precise pairing, such that stable and specific binding occurs between the oligonucleotide and the DNA or RNA target.

For purposes of this invention, “homology” or “homologous” refers to the percent homology between two polynucleotides, two nucleic acids sequences or two polypeptides. The correspondence between two sequences can be determined by techniques known in the art. In the context of the present invention two DNA or two polypeptide sequences are “substantially homologous” to each other when at least 15, preferably at least 17, preferably at least 18, more preferably at least 19, and most preferably at least 20 of the nucleotides or amino acids match over a defined length of the molecules, as determined using methods in the art. A substantially homologous sequence can compete for and inhibit the binding (the hybridization) of a completely homologous sequence to a target sequences under conditions of low stringency.

By “modulate” and “modulation” is meant that the expression of the gene, or level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the term “modulate” can mean “inhibit” and within the scope of the invention, the preferred form of modulation is inhibition but the use of the word “modulate” is not limited to this definition.

By “target site” is meant a sequence within a target RNA that is “targeted” or cleavage mediated by a siNA construct which contains sequences within its antisense region that are complementary to the target sequence.

By “inhibit” it is meant that the levels of expression product or level of RNAs or equivalent RNAs encoding one or more gene products is reduced below that observed in the absence of the nucleic acid molecule of the invention. In one embodiment, inhibition with a siNA molecule preferably is below that level observed in the presence of an inactive or attenuated molecule that is unable to mediate an RNAi response.

By “gene” or “target gene” is meant a nucleic acid that encodes an RNA, for example, nucleic acid sequences including, but not limited to, structural genes encoding a polypeptide. The target gene can be a gene derived from a cell, an endogenous gene, a transgene, or exogenous genes such as genes of a pathogen, for example a virus, which is present in the cell after infection thereof. The cell containing the target gene can be derived from or contained in any organism, most preferably an animal. Non-limiting examples of animals include vertebrates and invertebrates. In the context of the invention, “gene” or “target gene” is most preferably the p65 subunit of NF-kappa-B.

By “RNA” is meant a molecule comprising at least one ribonucleotide residue. By “ribonucleotide” is meant a nucleotide with a hydroxyl group at the 2′position of a-D-ribo-furanose moiety. The terms include double-stranded RNA, single-stranded RNA, isolated RNA such as partially purified RNA, essentially pure RNA, synthetic RNA, recombinantly produced RNA, as well as comprising non-standard nucleotides, such as non-naturally occurring nucleotides or chemically synthesized nucleotides or deoxynucleotides.

By “subject” is meant an organism, which is a donor or recipient of explanted cells or the cells themselves. “Subject” also refers to an organism to which the nucleic acid molecules of the invention can be administered. In one embodiment, a subject is a mammal or mammalian cells. In another embodiment, a preferred subject is a human subject or human cells.

The term “phosphorothioate” as used herein refers to an inter-nucleotide linkage comprising a sulfur atom in placement for an oxygen within the phosphate linkages of the sugar phosphate backbone. Hence, the term phosphorothioate refers to both phosphorothioate and phosphorodithioate inter-nucleotide linkages.

By “vectors” is meant any nucleic acid-and/or viral-based technique used to deliver a desired nucleic acid molecule inside a cell, biological system or organism.

By the term “non-nucleotide” is meant any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine and therefore lacks a base at the 1′-position.

By “nucleotide” as used herein, is as recognized in the art to include natural bases (standard), and modified bases well known in the art. There are several examples of modified nucleic acid bases known to a skilled person, such as those summarized by Limbach et al., 1994.

The term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule capable of mediating RNA interference “RNAi” or gene silencing in a sequence-specific manner.

The term “biodegradable linker” as used herein, refers to a nucleic acid or non-nucleic acid linker molecule that is designed as a linker molecule that is degradable by the surrounding milieu, inside a cell or organism and may be used to connect one molecule to another molecule, for example, a biologically active molecule to a siNA molecule of the invention or the sense and antisense strands of a siNA molecule of the invention.

The term “biologically active molecule” as used herein, refers to compounds or molecules that are capable of eliciting or modifying a biological response in a system

By “pharmaceutically acceptable formulation” is meant a composition or formulation that allows for the effective distribution of the nucleic acid molecules of the invention in that physical location most suitable for their desired activity.

By “systemic administration” is meant in vivo systemic absorption or accumulation of drugs in the blood stream followed by distribution throughout the entire body

By “biological system” is meant material, in a purified or non-purified form, from biological sources, including but not limited to human, animal, plant, insect, bacterial, viral or other sources, wherein the system comprises the components required for RNAi activity.

By “phenotypic change” is meant any detectable change to a cell that occurs in response to contact or treatment with a nucleic acid molecule of the invention (e.g., siNA). Such detectable changes include, but are not limited to, changes in shape, size, proliferation, motility, protein expression or RNA expression or other physical or chemical changes as can be assayed by methods known in the art. The detectable change can also include expression of reporter genes/molecules such as Green Florescent Protein (GFP) or lucifersase or various tags that are used to identify an expressed protein or any other cellular component that can be assayed.

### PREFERRED EMBODIMENTS OF THE INVENTION

One embodiment of the invention provides a short interfering RNA (siRNA) molecule that down regulates expression of the p65 subunit of NF-kappa-B by RNA interference. The siRNA molecule can be used to treat conditions associated with the expression of p65, for example cancer, allergic/inflammatory diseases and conditions, including but not limited to asthma, allergic rhinitis, atopic dermatitis, psoriasis, rheumatoid arthritis, ulcerative proctits, ulcerative colitis, Crohn's disease, septic shock, and other diseases that are NF-kappa-B dependent.

An siRNA molecule can comprises sense region and an antisense region and wherein said antisense region comprises sequence complementary to an RNA sequence encoding the p65 subunit of NF-kappa-B and the sense region comprises sequence complementary to the antisense region.

The sIRNA molecule can be assembled from two nucleic acid fragments wherein one fragment comprises the sense region and the second fragment comprises the antisense region of said siRNA molecule.

The target region within the mRNA sequence encoding the p65 subunit of NF-kappa-B is preferably chosen among the sequences presented as SEQ.ID.NOs, 1, 2, 3, and 4. The target sequences were identified in human NF-kappa-B transcription factor p65 subunit mRNA, GenBank accession number M62399. See Table 1.

TABLE 1

NF-kappa-B target and siRNA compound
sequences (5′-3′)

| | | |
|---|---|---|
| SEQ | | Applicant's |
| ID | | reference |
| NO | Sequence | no. |

Target Sequence

| | | |
|---|---|---|
| 1 | AAGGACCUAUGAGACCUUCAA | IDX 101 |
| 2 | AAGAUCAAUGGCUACACAGGA | IDX 105 |
| 3 | AACACUGCCGAGCUCAAGAUC | IDX 106 |
| 4 | GAGUCAGAUCAGCUCCUAAGG | IDX 107 |

Lower Sequence

| | | |
|---|---|---|
| 5 | UUGAAGGUCUCAUAGGUCC(D)TT* | IDX 121 |
| 6 | UCCUGUGUAGCCAUUGAUC(D)TT | IDX 125 |
| 7 | GAUCUUGAGCUCGGCAGUG(D)TT | IDX 126 |
| 8 | CCUUAGGAGCUGAUCUGAC(D)TT | IDX 127 |

Upper Sequence

| | | |
|---|---|---|
| 9 | GGACCUAUGAGACCUUCAA(D)TT* | IDX 131 |
| 10 | GAUCAAUGGCUACACAGGA(D)TT | IDX 135 |
| 11 | CACUGCCGAGCUCAAGAUC(D)TT | IDX 136 |
| 12 | GUCAGAUCAGCUCCUGGAA(D)TT | IDX 137 |

Notes:
*denotes that siRNA compound composed of antisense stand SEQ ID NO 5 annealed with sense strand SEQ ID NO 9 that was used in the animal proof of concept studies.

Compounds synthesized as RNA oligonucleotides with the last two nucleotides from the 3′ end are DNA, denoted as (D).

The 3′-ends of the Upper sequence and the Lower sequence of the siRNA construct can include an overhanging sequence, for example 1, 2, 3, or 4 nucleotides in length, preferably 2 nucleotides in length, wherein the overhanging sequence of the lower sequence is optionally complementary to a portion of the target sequence. The upper sequence is also referred to as the sense strand, whereas the lower sequence is also referred to as the antisense strand.

The target region within the mRNA sequence encoding the p65 subunit of NF-kappa-B can comprise of sequences of up to 60 contiguous nucleotides containing entirely any of SEQ ID NOs, 1, 2, 3 and 4 within that 60 contiguous nucleotide stretch.

The antisense region of the p65 subunit of NF-kappa-B siRNA compound can comprise a sequence complementary to sequence chosen among SEQ ID NOs. 5, 6, 7 and 8 or substantially homologous sequences thereof.

The sense region of the p65 subunit of NF-kappa-B siRNA compound can comprise a sequence complementary to sequence chosen among SEQ ID NOs 9, 10, 11 and 12 or substantially homologous sequences thereof.

The sense region of the p65 subunit of NF-kappa-B siRNA compound preferably comprises the sequence of SEQ ID NO. 9, the corresponding antisense region of the p65 subunit of NF-kappa-B siRNA compound being SEQ ID NO. 5.

The sense region of the p65 subunit of NF-kappa-B siRNA compound preferably comprises the sequence of SEQ ID NO. 10, the corresponding antisense region of the p65 subunit of NF-kappa-B siRNA compound being SEQ ID NO. 6

The sense region of the p65 subunit of NF-kappa-B siRNA compound preferably comprises the sequence of SEQ ID NO. 11, the corresponding antisense region of the p65 subunit of NF-kappa-B siRNA compound being SEQ ID NO. 7.

The sense region of the p65 subunit of NF-kappa-B siRNA compound preferably comprises the sequence of SEQ ID NO. 12, the corresponding antisense region of the p65 subunit of NF-kappa-B siRNA compound being SEQ ID NO. 8.

In one embodiment of the present invention the sense region and antisense region of the siNA and/or siRNA molecule are covalently connected via a linker molecule. The linker molecule can be a polynucleotide linker or a non-nucleotide linker.

Synthesis of nucleic acids greater than 100 nucleotides in length is difficult using automated methods, and the therapeutic cost of such molecules is prohibitive. In this invention, small nucleic acid motifs (“small” referring to nucleic acid motifs no more than 100 nucleotides in length, preferably no more than 80 nucleotides in length, and most preferably no more than 50 nucleotides in length; e.g., individual siNA oligonucleotide sequences or siNA sequences synthesized in tandem) are preferably used for exogenous delivery. The simple structure of these molecules increases the ability of the nucleic acid to invade targeted regions of protein and/or RNA structure.

The method of synthesis used for RNA including certain siNA molecules of the invention follows the procedure as described in Usman et al., 1987, J. Am. Chem. Soc., 109, 7845; Scaringe et al., 1990, Nucleic Acids Res., 18, 5433.

The introduction of chemically modified nucleotides into nucleic acid molecules provides a powerful tool in overcoming potential limitations of in vivo stability and bioavailability inherent to native RNA molecules that are delivered exogenously. For example, the use of chemically modified nucleic acid molecules can enable a lower dose of a particular nucleic acid molecule for a given therapeutic effect since chemically modified nucleic acid molecules tend to have a longer half-life in serum, such for example locked nucleic acids (LNA).

Furthermore, certain chemical modifications can improve the bioavailability of nucleic acid molecules by targeting particular cells or tissues and/or improving cellular uptake of the nucleic acid molecule. Therefore, even if the activity of a chemically-modified nucleic acid molecule is reduced as compared to a native nucleic acid molecule, for example, when compared to an all-RNA nucleic acid molecule, the overall activity of the modified nucleic acid molecule can be greater than that of the native molecule due to improved stability and/or delivery of the molecule. Unlike native unmodified siNA, chemically modified siNA can also minimize the possibility of activating interferon activity in humans.

In one embodiment of the invention the sense region of the siNA and/or siRNA molecule comprises a 3′-terminal overhang and the antisense region of the siRNA molecule comprises a 3′-terminal overhang. The 3′-terminal overhangs can each comprise 1 to 5 natural or modified nucleotides. In one embodiment the 3′-terminal nucleotide overhang of the antisense region is complementary to RNA encoding p65 subunit of NF-kappa-B.

In another embodiment of the invention the sense region of the siNA and/or siRNA molecule comprises one or more 2′-O-methyl modified pyrimidine nucleotides.

The antisense and/or region of a siNA molecule of the invention can comprise a phosphorothioate internucleotide linkage at the 3′-end of said antisense and/or sense region. The antisense and/or sense region can comprise about one to about five phosphorothioate internucleotide linkages at the 5′-end of said antisense region. The 3′-terminal nucleotide overhangs of a siNA molecule of the invention can comprise nucleotides or non-nucleotides. In particular, the 3′-terminal nucleotide overhangs of a siNA molecule of the invention can comprise ribonucleotides or deoxyribonucleotides that are chemically modified at a nucleic acid sugar, base, or backbone. The 3′-terminal nucleotide overhangs can comprise one or more universal base ribonucleotides. The 3′-terminal nucleotide overhangs can comprise one or more acyclic nucleotides. All modifications are well known in the art.

In one embodiment the sense strand of the siNA and/or the siRNA molecule of the invention comprises a terminal cap moiety at the 5′-end, 3′-end, or both 5′ and 3′ ends of said sense region. In another embodiment the antisense strand comprises one or more 2′-deoxy-2′-fluoro modified pyrimidine nucleotides.

For example, an exemplary chemically modified siNA molecule of the invention comprises a duplex having two strands, one or both of which can be chemically modified with a chemical modifications known in the art.

In another embodiment, any modified nucleotides present in the siNA molecules of the invention, preferably in the antisense strand of the siNA molecules of the invention, but also optionally in the sense and/or both antisense and sense strands, comprise modified nucleotides having properties or characteristics similar to naturally occurring ribonucleotides.

In another embodiment, the conjugate is covalently attached to the chemically modified siNA molecule via a biodegradable linker. In one embodiment, the conjugate molecule is attached at the 3′-end of either the sense strand, the antisense strand, or both strands of the chemically-modified siNA molecule.

In another embodiment, the conjugate molecule is attached at the 5′-end of either the sense strand, the antisense strand, or both strands of the chemically-modified siNA molecule. In yet another embodiment, the conjugate molecule is attached both the 3′-end and 5′-end of either the sense strand, the antisense strand, or both strands of the chemically-modified siNA molecule, or any combination thereof.

Such conjugates and/or complexes can be used to facilitate delivery of siNA molecules into a biological system, such as a cell. The conjugates and complexes indicated by the invention can impart therapeutic activity by transferring therapeutic compounds across cellular membranes, altering the pharmacokinetics, and/or modulating the localization of nucleic acid molecules of the invention. The use of such conjugates is well known in the art.

In one embodiment, the invention features a short interfering nucleic acid (siNA) molecule capable of mediating RNA interference (RNAi) inside a cell or reconstituted in vitro system, wherein one or both strands of the siNA molecule that are assembled from two separate oligonucleotides do not comprise any ribonucleotides. All positions within the siNA can include chemically modified nucleotides and/or non-nucleotides.

In another embodiment, the, siNA of the p65 subunit of NF-kappa-B gene can be used to characterize pathways of gene function in a variety of applications by monitoring phenotypic changes. For example, the present invention can be used to inhibit the activity of target gene (s) in a pathway to determine the function of uncharacterized gene (s) in gene function analysis, mRNA function analysis, or translational analysis. The invention can be used to determine potential target gene pathways involved in various diseases and conditions toward pharmaceutical development. The invention can be used to understand pathways of gene expression involved in, for example, inflammation and other diseases and disorders.

In one embodiment, the invention features a composition comprising a siNA molecule of the invention, which can be chemically modified, in a pharmaceutically acceptable carrier or diluent. In another embodiment, the invention features a pharmaceutical composition comprising siNA molecules of the invention, which can be chemically modified, targeting one or more genes in a pharmaceutically acceptable carrier or diluent.

In another embodiment, the invention features a method for treating, preventing or alleviating a disease or condition in a subject, comprising administering in a therapeutically effective amount and in a suitable pharmacological carrier a siRNA compound of the invention to the subject, most preferable a human, under conditions suitable for the treatment or prevention of the disease through which inhibition of the p65 subunit of NF-kappa-B is believed to be critical for the prevention of the disease or condition in the subject. The suppression and/or inhibition of the expression of the p65 subunit of NF-kappa-B suppress and/or inhibit NF-kappa-B dependent processes.

The terms “short interfering nucleic acid”, “siNA”, “short interfering RNA”, “siRNA”, “short interfering nucleic acid molecule”, “short interfering oligonucleotide molecule”, or “chemically modified short interfering nucleic acid molecule” as used herein refers to any nucleic acid molecule capable of mediating RNA interference (RNAi) or gene silencing in a sequence-specific manner.

The siNA molecules of the invention are added directly, or can be complexed with cationic lipids, packaged within liposomes, or otherwise delivered to target cells or tissues. The nucleic acid or nucleic acid complexes can be locally administered to relevant tissues ex vivo, or in vivo through injection, infusion pump or stent, with or without their incorporation in biopolymers.

In another aspect, the invention provides mammalian cells containing one or more siNA molecules of this invention. The one or more siNA molecules can independently be targeted to the same or different sites.

The nucleic acid molecules of the instant invention, individually, or in combination or in conjunction with other drugs, can be used to treat diseases or conditions discussed herein (e.g., inflammation diseases, cancers and other conditions in which NF-kappa-B plays a role). For example, to treat a particular disease or condition, the siNA molecules can be administered to a subject or can be administered to other appropriate cells evident to those skilled in the art, individually or in combination with one or more drugs under conditions suitable for the treatment.

One embodiment of the invention is a method of preventing, treating or alleviating NF-kappa-B dependent conditions in an individual, which comprises the extraction of cells, tissue or entire organs from said individual; contacting the said cells, tissue or entire organs with a siRNA compound according to the invention, so that expression of the p65 subunit of NF-kappa-B is suppressed, thereby suppressing NF-kappa-B dependent processes; and reintroducing the same cells, tissue or entire organ. Such method/methods may be used as a step in a treatment involving one of transplantation, graft, or implantation.

The siNA/siRNA molecules of the invention can be administered to an individual in a dose corresponding to 0.01 μg-100 mg/kg body weight, preferably 0.01 μg-10 mg/kg body weight, more preferably 0.01 μg-1 mg/kg body weight, and most preferably 0.01 μg-0.1 mg/kg body weight.

In a further embodiment, the siNA molecules can be used in combination with other known treatments to treat conditions or diseases discussed above. For example, the described molecules could be used in combination with one or more known therapeutic agents to treat a disease or condition. Non-limiting examples of other therapeutic agents that can be readily combined with a siNA molecule of the invention are enzymatic nucleic acid molecules, allosteric nucleic acid molecules, antisense, decoy, or aptamer nucleic acid molecules, antibodies such as monoclonal antibodies, small molecules, and other organic and/or inorganic compounds including metals, salts and ions.

One embodiment of the invention provides an expression vector comprising a nucleic acid sequence encoding at least one siNA and/or siRNA molecule of the invention in a manner that allows expression of the nucleic acid molecule. Another embodiment of the invention provides a mammalian cell comprising such an expression vector. The mammalian cell can be a human cell. The siNA and/or siRNA molecule of the expression vector can comprise a sense region and an antisense region. The antisense region can comprise sequence complementary to a RNA or a DNA sequence encoding the p65 subunit of NF-kappa-B and the sense region can comprise sequence complementary to the antisense region. The siNA molecule can comprise two distinct strands having complementary sense and antisense regions. The siNA molecule can comprise a single strand having complementary sense and antisense regions.

In one embodiment, the invention features an expression vector comprising a nucleic acid sequence encoding at least one siNA molecule of the invention, in a manner, which allows expression of the siNA molecule. For example, the vector can contain sequence (s) encoding both strands of a siNA molecule comprising a duplex. The vector can also contain sequence (s) encoding a single nucleic acid molecule that is self-complementary and thus forms a siNA molecule.

The recombinant vectors capable of expressing the siNA molecules can be delivered as described herein, and persist in target cells. Alternatively, viral vectors can be used that provide for transient expression of siNA molecules. Such vectors can be repeatedly administered as necessary. Once expressed, the siNA molecules bind and down-regulate gene function or expression via RNA interference (RNAi). Delivery of siNA expressing vectors can be systemic, such as by intravenous or intramuscular administration, by administration to target cells ex-planted from a subject followed by reintroduction into said subject, or by any other means that would allow for introduction into the desired target cell.

Other features and advantages of the invention will be apparent from the description of the preferred embodiments thereof, and from the claims.

### EXAMPLES

The following are non-limiting examples showing the selection, isolation, synthesis and activity of nucleic acids of the instant invention.

### Example 1

### Construction of siNA Compounds

In a non-limiting example, RNA oligonucleotides were synthesized in a stepwise fashion using the phosphoramidite chemistry known in the art.

All RNAs were purchased from Eurogentec Ltd (Liège, Belgium), and received as desalted powder. The sequence of RNAs is given in Table 1, wherein “upper sequence” denotes the sense sequence and “lower sequence” denotes the antisense sequence. The last two nucleotides from the 3′end are synthesized as DNA nucleotides. The powder for each siNA molecule was dissolved in 100 mM potassium acetate, 30 mM HEPES-KOH, 2 mM magnesium acetate, pH 7.4 at a concentration of 100 μM. The siRNA duplexes were formed by mixing equimolar concentrations at 50 μM of SEQ ID NO. 9 with SEQ.ID.NO. 5, giving rise to compound IDX 0131, SEQ ID NO. 10 with SEQ ID NO. 6, giving rise to compound IDX 0135, SEQ ID NO. 11 with SEQ ID NO. 7 giving rise to compound IDX 0136, and SEQ ID NO. 12 with SEQ ID NO. 8 giving rise to compound IDX 0137. See also Table 1.

The mixed samples were then heated to 90° C. for 1 minute and incubated at 37° C. for one hour to allow the two strands to anneal to each other. Each solution of annealed pairs was diluted to 20 μM, aliquoted and stored frozen at −20° C. until further use.

### Example 2

### Selection of Potential siNA Target Sites in the RNA Sequence of P65 Subunit of NF-kappa-B

The sequence of an RNA target of interest, such as a viral or human mRNA transcript, is screened for target sites, for example by using a computer folding algorithm. In a non-limiting example, the sequence of a gene or RNA gene transcript derived from a database, such as Genbank, is used to generate siNA targets having complementarity to the target. Such sequences can be obtained from a database, or can be determined experimentally as known in the art. Target sites that are known, for example, those target sites determined to be effective target sites based on studies with other nucleic acid molecules, for example ribozymes or antisense, or those targets known to be associated with a disease or condition such as those sites containing mutations or deletions, can be used to design siNA molecules targeting those sites.

Various parameters can be used to determine which sites are the most suitable target sites within the target RNA sequence. These parameters include but are not limited to secondary or tertiary RNA structure, the nucleotide base composition of the target sequence, the degree of homology between various regions of the target sequence, comparison of homology between various regions of the target sequence or highly conserved sequence regions derived from different species such as mouse rat or human, or indeed the relative position of the target sequence within the RNA transcript.

However, while there exists prediction methods, for example using a computer folding algorithms, that allow identification of a potential siNA target site within a target transcript, these methods provide no indication as to whether such sites are effective in achieving RNAi mediated inhibition of the target gene. As a consequence, considerable experimentation and an inventive approach is required to identify sites that demonstrate efficacy in a biological system.

The present inventor surprisingly identified four novel target sites implicated in NF-Kappa-B subunit p65 expression and developed active and specific siNA molecules to these sites.

The following non-limiting steps can be used to carry out the selection of siNAs targeting the p65 subunit of NF-kappa-B mRNA transcript.

A collection of sequence specific siNA compounds designed to target human p65 subunit of NF-kappa-B mRNA where screened for efficacy in cells that contained a luciferase reporter construct.

The use of such luciferase reporter constructs is well know in the art and provides a simple means to determine the degree of activity of a particular gene of interest depending on the design of the construct used. In the scope of the present invention the particular construct employed was designed such that it was sensitive to the levels of endogenous active NF-kappa-B. In other words, the construct contained typical “NF-kappa-B binding sites” as discrete genetic elements such that active NF-kappa-B would bind to said elements and induce the expression of the reporter gene luciferase. Higher levels of NF-kappa-B would be indicative of higher levels of luciferase activity within a cell. Conversely, lower levels of NF-kappa-B would be indicated of lower levels of luciferase activity.

It follows, that by introduction into such cells harboring the luciferase construct, of the siRNA compounds designed to target the mRNA encoding the p65 subunit of NF-kappa-B, those compounds that demonstrate efficacy will result in decreased levels of mRNA encoding the p65 subunit of NF-kappa-B as mediated by the RNAi effect and therefore decreased levels of p65 protein. As NF-kappa-B exists and functions as a heterodimer of two proteins, namely p50 and p65, reduction in the levels of p65 protein will result in reduced NF-kappa-B activity. This reduction can be monitored using said luciferase reporter constructs.

The target sequences corresponding to SEQ ID NOs. 9, 10, 11 and 12 were identified, as well as the antisense sequences corresponding to SEQ ID NOs. 5, 6, 7 and 8 developed by the present inventors.

Cells expressing the luciferase construct (e.g., 293 cells) are transfected with the collection of siNA compounds (ID.NO IDX 0131 and IDX 0121; ID.NO IDX 0135 and IDX 0125; ID.NO IDX 0136 and IDX 0126; and ID.NO IDX 0137 and IDX 0127 and the levels of luciferase activity is monitored. In detail, on the day before transfection, 293 cells were harvested and distributed into 24 well plates at 0.1×106 cells per well. On day 0, cells were transfected with siRNA as follows (description refers to a single group of transfection) 4.5 ul of oligofectamine was diluted in 18 ul of Optimem I medium (Invitrogen) and incubated for 5 min at room temperature 7.5 ul of respective siRNA (20 uM) were diluted in 120 ul of Optimem I medium and directly afterwards were mixed with Oligofectamine solution. Oligofectamine/siRNA complexes were formed for 20 minutes at room temperature.

During this incubation cell culture medium was replaced to 200 ul of serum-free Minimal essential medium per well (4 well per group). Medium exchange was done by removing medium with 1 ml tip and then directly adding serum-free medium with another pipette to minimize drying out.

Oligofectamine/siRNA complexes were added at 50 ul per well. Thereafter cells were incubated in transfection mixture for 6 hours at 37° C. supplemented with 5% CO2. On day 2 after transfection, cells from 2 wells were trypsinized and transferred to 3 wells in 24 well cultures. On day 3 cells were transfected with a cocktail containing 0.01 ug NF-kappa-B reporter plasmid and 0.03 ug/well of beta-galactosidase plasmid. Transfection was performed using Fugene 6 (Roche) as a transfection agent at the ratio 2 ug of DNA/3 ul of Fugene 6 and following manufacturer's instructions. On day 4 (16 hours after reporter transfection) 25 ng/ml of TNF were added to stimulate the cell cultures.

On reporter assays were performed after 24 hours following transfection on day 5, using Luciferase detection part of Dual-Luciferase reporter system (Promega).

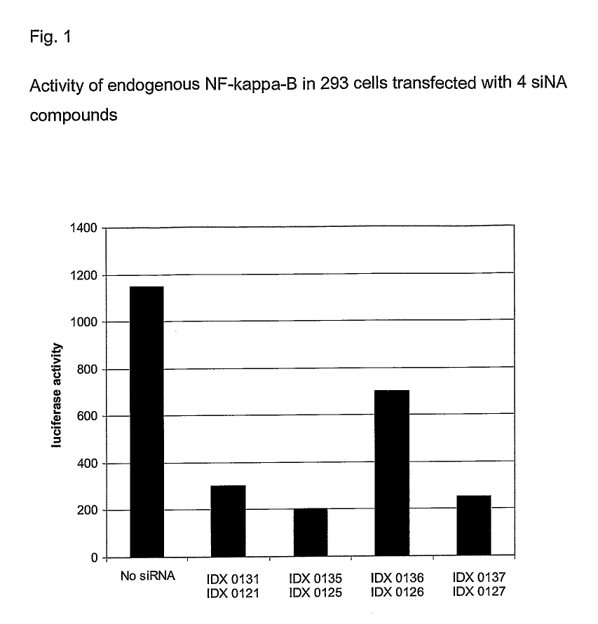

As is evident from FIG. 1, the cells that received no siRNA compound show a high luciferase activity, whereby cells that were transfected with siRNA compounds IDX 0131/IDX 0121, IDX 0135/IDX 0125, IDX 0136/IDX 0126 and IDX 0137/IDX 0127 demonstrate functional siRNA compounds in that the activity of luciferase is reduced. This is most markedly seen with compound IDX 0135, the rank order of potency being IDX 0135>IDX 0137>IDX 0131>IDX 0136. Transfection of cells using siRNA compounds of unrelated sequences caused only a very small reduction of luciferase activity and can be largely attributed to unspecific effects a phenomenon that has been observed and reported by many researchers in the field.

### Example 3

### Determination of Inhibitor Effects In Vitro of the siRNA Compounds on the p65 Subunit of NF-kappa-B

Cells were treated as described in Example 1. On day 4, the medium was removed and cells were lysed in 50 μl of lysis buffer (20 mM Tris pH 7.5, 1% TritonX-100, 1 mM EDTA, 150 mM NaCl, 1 mM EDTA, 1 mM PMSF, Aprotinin and Leupeptin) for 20 minutes on ice. Thereafter, lysates were transferred to 1.5 ml Expenders tubes and centrifuged for 10 min at 14,000 rpm to remove cellular debris and nuclei. Thereafter, 40 μl of lysates were combined with 40 μl of reduced Laemli sample buffer for SDS-PAGE and incubated at 95° C. for 5 min. 20 μl of each sample were loaded per well. After electrophoresis, proteins were blotted to nitrocellulose membranes using mini-cell blotting apparatus (Biorad). The membranes were blocked in 5% non-fat milk and then were incubated with antibodies directed to p65 (Santa Cruz, sc-372) for 1 hour followed by HRP-coupled anti-rabbit reagent (Amersham Biosciences, NA934V) for one hour.

Thereafter membranes were developed using ECL detection reagent (Amersham Biosciences, RPN2106V1).

All antibodies were applied diluted 1 to 1000 in Tris-buffered saline (pH 7.5) containing 5% of non-fat dry milk and 0.05% Tween-20. To confirm equal loading, membranes were subsequently incubated with antibodies to human b-actin (Santa Cruz, I-19) and HRP-coupled rabbit anti-goat antibodies (DAK0, P0449) using procedures described above.

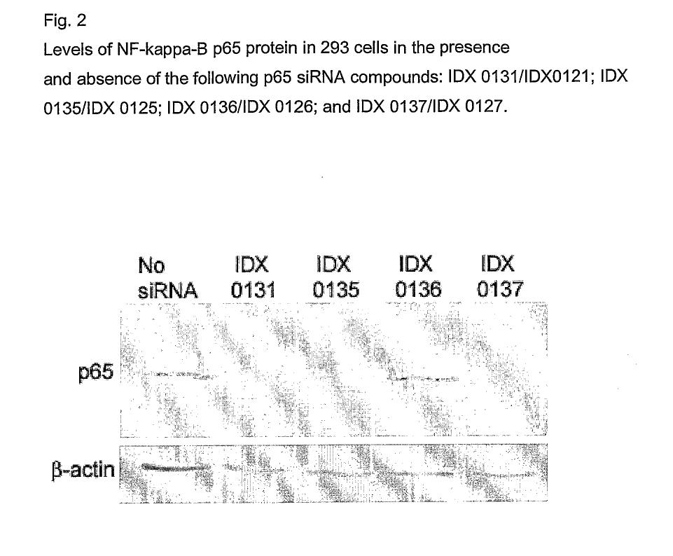

From FIG. 2 it is evident that siRNA compounds IDX 0131/IDX 0121; IDX 0135/IDX 0125; and IDX 0137/IDX 0127 are able to down regulate the expression of the p65 subunit of NF-kappa-B protein to levels of near non detection when compared to levels of p65 protein in cells in the absence of siRNA compound. In contrast, siRNA compound IDX 0136/IDX 0126 was only able to produce a moderate down regulation of the p65 protein level. These results are in good agreement to those described in Example 2.

### Example 4

### Suppression of Inflammation in a Colitis Mouse Model Using siRNA Compound IDX 0131/DX 0121 Designed to Inhibit NF-kappa-B p65 Subunit

An animal model wherein inflammation is induced in the large intestine of mice has been described by Okayasu et al., 1990. In the model used in the present experiment, oral dextran sulfate sodium (DSS) was employed to induce inflammation (Axelsson, et al., 1998). DSS can be given to the mice in the drinking water, thereby inducing a colitis resembling inflammatory bowel disease (IBD) in man. An MW of about 40-50 kD and an high content of up to about 19% sulphur has been shown to be optimal for the inflammation inducing form of DSS. In Okayasu, 1990, the DSS was given to the animals at a concentration of about 2-5%.

In this study DSS was used at a concentration of 2.5%, dissolved in water, with a final pH of 8.5 (adjusted with NaOH). DSS was given orally to female SPF NMRI mice for 8 consecutive days to induce a stable colitis in all individuals. This type of experimentally induced colitis has been shown to be fully induced at day 4-5 after addition in the drinking water (Cooper et al., 1993).

The siRNA substance, as given by IDX 0131/IDX 0121 was administered rectally to non-medicated or anaesthetized colitic animals. A shortened XRO feeding tube (Vygon, Ecouen, France) was inserted rectally, up to the level of the ligament of Treitz, and the substance, in a final volume of 100 ul, was administered during slow careful retraction of the tubing to avoid rectal leakage of the substance.

Two groups of animals were used in this study, one group received a single dose of 10 uM of siRNA compound IDX 0131/IDX 0121 comprising SEQ ID NO. 5 and SEQ ID NO. 9 in 100 ul of water. The other group received a single dose of 40 uM of siRNA compound IDX 0131/IDX 0121 comprising SEQ ID NO. 5 and SEQ ID NO. 9 in 100 ul water.

Therapeutic treatment was given once on day 8 while the DSS treatment continued another 10 days. On day 18 the animals were killed and subjected to analysis of clinical inflammatory parameters and histopathological examinations.

Clinical Signs

Each mouse was observed once daily during the study period. All signs of bad health and any behavioral changes were recorded. Animals showing severe signs of disease and losing more than 15% of its original body weight were killed.

Mortality and Necropsy

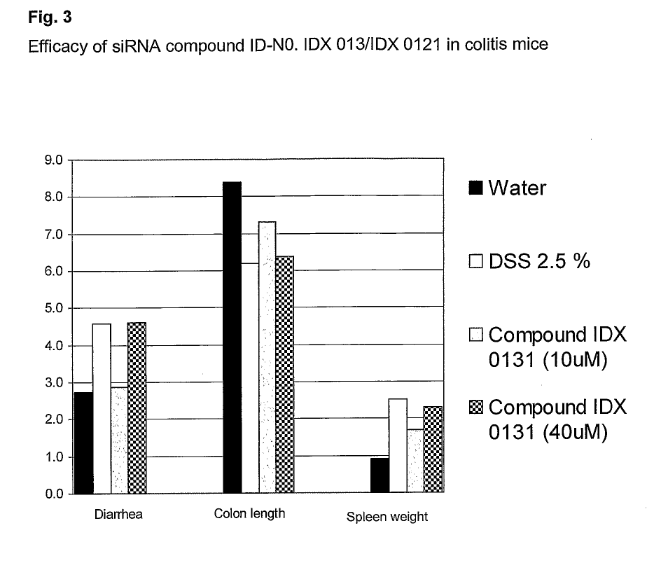

Mortality during the experimental period was recorded. At the end of the experimental period, animals were killed by dislocation of the cervical spine. The abdomen was opened and the spleen was resected and weighed. The large intestine was excised from the ileocecal junction to the proximal rectum, close to its passage under the pelvisternum. The caecum was opened at the apex and feces were carefully removed. The colon was opened longitudinally and the feces were carefully removed with a spatula. Evaluation of colitis was made by recording clinical parameters such as mortality, colon length, spleen weight and diarrhea, calculated as wet/dry weight of the feces after drying 48 h at 60° C. (FIG. 3). The entire caecum and colon were fixed in 4% neutral buffered formaldehyde for microscopic examination.

It is evident from FIG. 3 that there is a specific and significant improvement in all measured parameters. That is to say, treated animals had less diarrhea, had a more normal colon length, a more normal spleen weight and showed statistically significant signs of histological improvement.

Processing and Microscopic Examination

After fixation, the tissues sampled for microscopic examination were trimmed and specimens were taken from caecum and the mid portion of colon for histological processing. Additional specimens were taken when the first sample was difficult to interpret. The specimens were embedded in paraffin and cut at a nominal thickness of 5 μm, stained with haematoxylin and eosin, and examined under light microscope.

Verification of colitis and estimation of inflammation was performed by an experienced veterinary pathologist, having extensive experience of the histopathological evaluation of DSS-induced colitis in mice. Diagnostic histopathology is based on a standardized grading system shown in Table 2.

TABLE 2
Histopathologic grading system
Colitis lesions:

| | |
|---|---|
| +/− | very mild (may be normal) |
| + | mild |
| ++ | moderate |
| +++ | severe |
| ++++ | very severe |

Histological Analysis of Colonic Sections

As outlined above, sections taken from the caecum and the mid portion of the colon were used for histological processing. Staining was performed with haematoxylin and eosin. Sections were then examined by light microscopy and morphological changes noted.

From FIG. 3 it can be concluded that a single rectal administration of antisense compound as given by compound IDX 0131/IDX 0121 comprising SEQ ID NO 5 and SEQ ID NO. 9 was sufficient to dramatically reduce the degree of inflammation as seen on all three physiological parameters.

In FIG. 3, a black solid bar denotes healthy animals that received only standard drinking water (healthy control). The unfilled bar denotes colitis induced animals who receive 2.5% DSS in their drinking water which will induce inflammation of the colon (sick control). The light grey bar denotes those animals that received in addition to DSS in their drinking water, siRNA compound IDX 0131/DX 0121 at a final concentration of 10 uM. Lastly, the dark grey bar denotes those animals who received in addition to DSS in their drinking water, siRNA compound IDX 0131/IDX 0121 at a final concentration of 40 uM. The beneficial effects seen with higher concentrations of siRNA compound IDX 0131/IDX 0121 are less pronounced and indicate a possible therapeutic threshold lower than 40 uM.

It is evident that a single rectal administration of siRNA compound as given by compound IDX 0131/IDX 0121 demonstrated clear signs of clinical improvement in colitis mice. This is most evident at a compound concentration of 10 uM with levels of statistical relevance of (P<0.005).

With respect to all three parameters, a dose of 10 uM siRNA compound IDX 0131/IDX 0121 improved dramatically the degree of diarrhoea, colon length and spleen weight to values near those seen in healthy controls (compare black bars to light grey bars).

The most effective dose appeared to be the lower of the two used in the study and indicate a possible therapeutic threshold of lower than 40 uM. Moreover, it is indeed well know in the art that NF-kappa-B while critical for the development and maintenance of an inflammatory phenotype is equally important in processes or rebuilding of damaged tissue as a result of inflammation. Consequently, there may be a need to establish the correct amount of inhibition of NF-kappa-B.

These results provide strong proof that by reducing the levels of NF-kappa-B beneficial effects regarding course and degree of inflammation are achieved. Moreover, the experiment also demonstrates that such diseases and indeed other diseases whereby NF-kappa-B is a critical factor in the pathogenesis of such diseases can be addressed using siRNA technology.

All patents and publications mentioned in the specification are indicative of the levels of skill of those skilled in the art to which the invention pertains. All references cited in this disclosure are incorporated by reference to the same extent as if each reference had been incorporated by reference in its entirety individually.

One skilled in the art would readily appreciate that the present invention is well adapted to carry out the objects and obtain the ends and advantages mentioned, as well as those inherent therein. The methods and compositions described herein as presently representative of preferred embodiments are exemplary and are not intended as limitations on the scope of the invention. Changes therein and other uses that will occur to those skilled in the art, which are encompassed within the spirit of the invention, are defined by the scope of the claims.

In addition, where features or aspects of the invention are described in terms of Markush groups or other grouping of alternatives, those skilled in the art will recognize that the invention is also thereby described in terms of any individual member or subgroup of members of the Markush group or other group.

### REFERENCES

Axeisson, et al., 1998, Aliment Pharmacol Ther, 12:925-34
Beach et al., WO 01/68836
Cooper, et al, 1993, Lab Invest, 69:238-49
Jensen, et al., 1999, Nat Genet 21:209-12
Ketting, et al., 1999, Cell, 99:133-41
Kreutzer et al., WO 00/44895
Limbach et al., 1994, Nucleic Acids Res. 22, 2183.
McSwiggen and Beigelman, WO 03/070970 A2
McSwiggen and Beigelman, WO 03/070918 A2
Okayasu, et al., 1990, Gastroenterology, 98:694-702
Perkins and Anderson, WO 03/020754 A2
Ratcliff, et al., 1999, Plant Cell, 11:1207-16
Savage J. et al., 2003, Proceedings of the American Association for Cancer Research, vol. 44, 2^{nd} ed., p. 449
Scaringe et al., 1990, Nucleic Acids Res., 18, 5433.
Surabhi R. M. and Gaynor R. B., 2002, J. Virol. Vol. 76:12963-12973
Tabara, et al., 1999, Cell 99:123-32
Usman et al., 1987, J. Am. Chem. Soc., 109, 7845
Zhou A. et al., 2003; Oncogene, 22:2054-2064

1. A short interfering RNA (siRNA) molecule that down regulates expression of a p65 subunit of NF-kappa-B gene by RNA interference, said siRNA molecule comprising a sense region and an antisense region and wherein said antisense region comprises a sequence complementary to an RNA sequence encoding the p65 subunit of NF-kappa-B and the sense region comprises a sequence complementary to the antisense region, characterized in that said antisense region comprises a sequence substantially complementary to a sequence chosen among SEQ ID NOs. 1, 2, 3 and 4 and wherein said antisense region comprises a sequence chosen among SEQ ID NOs. 5, 6, and 8 or substantially homologous sequences thereof.

2. The siRNA molecule of claim 1, wherein said sense region comprises a sequence chosen among SEQ ID NOs 9, 10, and 12 or substantially homologous sequences thereof.

3. The siRNA molecule of claim 1, wherein said sense region and antisense region are covalently connected via a linker molecule.

4. The siRNA molecule of claim 1, wherein said linker molecule is a polynucleotide linker.

5. The siRNA molecule of claim 1, wherein said linker molecule is a non-nucleotide linker.

6. The siRNA molecule of claim 1, wherein said sense region comprises the sequence of SEQ ID NO. 9 and said antisense region comprises the sequence of SEQ ID NO. 5.

7. The siRNA molecule of claim 1, wherein said sense region comprises the sequence of SEQ ID NO. 10 and said antisense region comprises a sequence of SEQ ID NO. 6.

8. The siRNA molecule of claim 1, wherein said sense region comprises the sequence of SEQ ID NO. 12 and said antisense region comprises the sequence of SEQ ID NO. 8.

9. The siRNA molecule of any one of claims 1-8, wherein said sense region comprises a 3′-terminal overhang and said antisense region comprises a 3′-terminal overhang.

10. The siRNA molecule of claim 9, wherein said 3′-terminal overhangs each comprising 1 to 5 natural or modified nucleotides.

11. The siRNA molecule of claim 9, wherein said antisense region 3′-terminal nucleotide overhang is complementary to RNA encoding p65 subunit of NF-kappa-B.

12. The siRNA molecule of claim 1, wherein said sense region comprises one or more 2′-O-methyl modified pyrimidine nucleotides.

13. The siRNA molecule of claim 1, wherein said sense strand comprises a terminal cap moiety at the 5′-end, 3′-end, or both 5′ and 3′ ends of said sense region.

14. The siRNA molecule of claim 1, wherein said antisense strand comprises one or more 2′-deoxy-2′-fluoro modified pyrimidine nucleotides.

15. The siRNA molecule of claim 1, wherein said antisense and/or sense strand comprises between one and up to and including five phosphorothioate internucleotide linkages at the 3′ end of said antisense region.

16. The siRNA molecule of claim 1, wherein said antisense and/or sense strand comprises between one and up to and including five phosphorothioate internucleotide linkages at the 5′ end of said antisense region.

17. The siRNA molecule of claim 9, wherein said 3′-terminal nucleotide overhangs comprise ribonucleotides that are chemically modified at a nucleic acid sugar, base, or backbone.

18. The siRNA molecule of claim 9, wherein said 3′-terminal nucleotide overhangs comprise deoxyribonucleotides that are chemically modified at a nucleic acid sugar, base, or backbone.

19. The siRNA molecule of claim 9, wherein said 3′-terminal nucleotide overhangs comprise one or more universal base ribonucleotides.

20. The siRNA molecule of claim 9, wherein said 3′-terminal nucleotide overhangs comprise one or more acyclic nucleotides.

21. The siRNA molecule of claim 9, wherein said 3′-terminal nucleotide overhangs comprise nucleotides or non-nucleotides

22. An expression vector comprising a nucleic acid sequence encoding at least one siRNA molecule of claim 1 in a manner that allows expression of the nucleic acid molecule.

23. A mammalian cell comprising the expression vector of claim 22.

24. The mammalian cell of claim 23, wherein said mammalian cell is a human cell.

25. The expression vector of claim 22, wherein said siRNA molecule comprises a sense region and an antisense region and wherein said antisense region comprises sequence complementary to an RNA sequence encoding p65 subunit of NF-kappa-B and the sense region comprises sequence complementary to the antisense region.

26. The expression vector of claim 22, wherein said siRNA molecule comprises two distinct strands having complementarity sense and antisense regions.

27. The expression vector of claim 22, wherein said siRNA molecule comprises a single strand having complementary sense and antisense regions.

28. A method of preventing, treating or alleviating NF-kappa-B dependent conditions in an individual, which comprises administrating a therapeutically effective amount and in a suitable pharmacological carrier, a siRNA compound of claim 1, so that expression of the p65 subunit of NF-kappa-B is suppressed, thereby suppressing NF-kappa-B dependent processes.

29. The method of claim 28, wherein the NF-kappa-B dependent condition is selected from cancer, cardiac disorders, ischaemia, allergic/inflammatory diseases and conditions, including but not limited to asthma, allergic rhinitis, atopic dermatitis, psoriasis, rheumatoid arthritis, ulcerative proctits, ulcerative colitis, Crohn's disease, septic shock, and other diseases or conditions that are NF-kappa-B dependent.

30. A method of preventing, treating or alleviating NF-kappa-B dependent conditions in an individual, which comprises the extraction of cells, tissue or entire organs from said individual; contacting the said cells, tissue or entire organs with a siRNA compound of claim 1, so that expression of the p65 subunit of NF-kappa-B is suppressed, thereby suppressing NF-kappa-B dependent processes; and reintroducing the same.

31. The method of claim 30, wherein said method is used as a step in a treatment involving one of transplantation, graft, or implantation.


SEQUENCE LISTING (12 sequences)

SEQ ID NO: 1 - RNA, 21 nt, Homo sapiens
  misc_RNA (1)...(21): IDX 101
aaggaccuaugagaccuucaa

SEQ ID NO: 2 - RNA, 21 nt, Homo sapiens
  misc_RNA (1)...(21): IDX 105
aagaucaauggcuacacagga

SEQ ID NO: 3 - RNA, 21 nt, Homo sapiens
  misc_RNA (1)...(21): IDX 106
aacacugccgagcucaagauc

SEQ ID NO: 4 - RNA, 21 nt, Homo sapiens
  misc_RNA (1)...(21): IDX 107
gagucagaucagcuccuaagg

SEQ ID NO: 5 - DNA, 21 bp, Homo sapiens
  misc_RNA (1)...(21): IDX 121
uugaaggucucauaggucctt

SEQ ID NO: 6 - DNA, 21 bp, Homo sapiens
  misc_RNA (1)...(21): IDX 125
uccuguguagccauugauctt

SEQ ID NO: 7 - DNA, 21 bp, Homo sapiens
  misc_RNA (1)...(21): IDX 126
gaucuugagcucggcagugtt

SEQ ID NO: 8 - DNA, 21 bp, Homo sapiens
  misc_RNA (1)...(21): IDX 127
ccuuaggagcugaucugactt

SEQ ID NO: 9 - DNA, 21 bp, Homo sapiens
  misc_RNA (1)...(21): IDX 131
ggaccuaugagaccuucaatt

SEQ ID NO: 10 - DNA, 21 bp, Homo sapiens
  misc_RNA (1)...(21): IDX 135
gaucaauggcuacacaggatt

SEQ ID NO: 11 - DNA, 21 bp, Homo sapiens
  misc_RNA (1)...(21): IDX 136
cacugccgagcucaagauctt

SEQ ID NO: 12 - DNA, 21 bp, Homo sapiens
  misc_RNA (1)...(21): IDX 137
gucagaucagcuccuggaatt